(19) United States
(12) Patent Application Publication
(10) Pub. No.: US 2026/0341402 A1
(43) Pub. Date: Oct. 29, 2026

(54) METHODS AND SYSTEMS FOR PROBABILISTIC SINGLE-SPERM GENOME RECONSTRUCTION AND SELECTION

(71) Applicant: Herasight Inc., Wilmington, DE (US)

(72) Inventors: Michael CHRISTENSEN, Wilmington, DE (US); Tobias WOLFRAM, Wilmington, DE (US)

(21) Appl. No.: 19/657,324

(22) Filed: Apr. 24, 2026

Related U.S. Application Data
(60) Provisional application No. 63/793,858, filed on Apr. 24, 2025.

(51) Int. Cl. G16B 20/20; G01N 33/53

(52) U.S. Cl. CPC G16B 20/20; G01N 33/5308

(57) ABSTRACT

Provided herein are methods and systems for designing and producing allele-discriminating reagent panels, determining genome-wide statistical trait predictions from a live sperm cell incubated with allele-discriminating reagent panels, and indexing and storing live sperm cells for downstream applications.

### CROSS-REFERENCE TO RELATED APPLICATIONS

This application claims the benefit of U.S. Provisional Patent Application No. 63/793,858, filed on Apr. 24, 2025, the entire disclosure of which is incorporated herein by reference to the extent permitted by law and to the extent not inconsistent with the present disclosure.

### FIELD OF INVENTION

The present disclosure relates to reproductive genetics and assisted reproduction, and more particularly to methods and systems for genetic characterization and selection of individual sperm prior to fertilization using multiplex allele-discriminating reagents, optical detection, probabilistic haplotype reconstruction, and genome-wide trait estimation.

### BACKGROUND

Preimplantation genetic testing has become an increasingly utilized component of assisted reproductive technology. Existing approaches may evaluate whole-chromosome aneuploidy, structural rearrangements, and monogenic disorders at the embryo stage, and more recent methods may further estimate genome-wide statistical trait predictors for complex traits or disease risk. These approaches generally rely on embryo culture followed by embryo biopsy and downstream genotyping or sequencing. As a result, such approaches are typically performed only after fertilization and may add cost, time, laboratory complexity, and attrition to the reproductive workflow.

Although embryo-stage testing can provide useful information, it occurs relatively late in the reproductive pipeline. Earlier-stage approaches that permit genetic assessment before fertilization, including at the level of individual gametes, have the potential to increase efficiency in assisted reproductive workflows by permitting selection of candidate sperm before intracytoplasmic sperm injection, in vitro fertilization, cryopreservation, or other downstream use.

Human sperm chromatin presents challenges for hybridization-based interrogation because much of the paternal genome is highly compacted and may not be equally accessible to probe-based detection. Accordingly, a practically useful pre-fertilization sperm assay may benefit not only from informative marker selection in recombination space, but also from accessibility-aware marker selection and signal-generation strategies that can produce measurable optical output from selected loci while preserving downstream usability of the sperm.

Existing sperm-selection and sperm-analysis systems do not provide a fully integrated platform for genome-scale prediction from sparse marker measurements made directly or indirectly on live sperm. In particular, conventional systems do not disclose a coordinated workflow that selects donor-specific heterozygous markers based on haplotype information content, recombination structure, and expected chromatin accessibility; uses allele-discriminating reagents to generate genotype-associated optical signal from those markers in live sperm; performs real-time optical measurement and gating on individual sperm based on fluorescence vectors or related optical measurements; sorts accepted sperm into indexed collection locations linked to digital records; and reconstructs genome-wide paternal genetic content probabilistically using recombination-aware and error-aware inference.

There is accordingly a need for methods and systems that enable genetic assessment and selection at the gamete stage, including selection based on one or more genome-wide statistical trait predictors and exclusion of specified genetic states, while remaining compatible with existing assisted reproductive procedures.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Provided herein are methods for determining a genome-wide statistical trait prediction from a sperm cell. The method may include obtaining a primary aliquot of a live sperm sample; incubating the primary aliquot with an allele-discriminating reagent panel to produce a labeled sperm, wherein the allele-discriminating reagent panel comprises a plurality of allele-discriminating reagents respectively directed to heterozygous markers selected based on phased paternal genomic information, a male recombination map, and one or more constraints; detecting an optical signal from the labeled sperm using an optical interrogation instrument; determining an optical vector based on the optical signal; sorting the labeled sperm to an indexed collection location, wherein the indexed collection location preserves an association between the labeled sperm and a digital record including the optical vector; determining a posterior probability distribution for the labeled sperm based on the optical vector and one or more prior inputs; and determining a genome-wide statistical trait prediction based on the posterior probability distribution.

In some aspects, the one or more constraints may include a chromatin-accessibility prior, a fluorescence-channel constraint, a recombination-based spacing constraint, or a combination thereof. In some aspects, the allele-discriminating reagent panel may include an allele-discriminating reagent comprising a target-binding region configured to bind to a target locus, and wherein the allele-discriminating reagent is configured to generate an optical signal. In some aspects, sorting the labeled sperm may include determining a gating computation for the labeled sperm, and wherein the gating computation is score-based or pattern-based. In some aspects, the indexed collection location may include a micro-well plate, a nano-well array, a droplet or barcoded droplet format, or a dielectrophoretic array. In some aspects, determining the posterior probability distribution may include using a Hidden Markov Model or an equivalent probabilistic graphical model. The one or more prior inputs may include a channel-specific error parameter, an accessibility parameter, paternal haplotype information, or a combination thereof.

In some aspects, the method may further include predicting an expected allele dosage at a plurality of loci based on the posterior probability distribution. In some aspects, the method may further include determining a confidence interval or uncertainty estimate for the genome-wide statistical trait prediction. In some aspects, the method may further include selecting the labeled sperm for fertilization according to a utility function that ranks the genome-wide statistical trait prediction.

Further provided herein are systems for determining a genome-wide statistical trait prediction from a sperm cell. The system may include an optical interrogation instrument configured to detect an optical signal from a labeled sperm and to generate an optical vector based on the optical signal, wherein the labeled sperm is produced by incubating a sperm cell with an allele-discriminating reagent panel comprising a plurality of allele-discriminating reagents respectively directed to a set of heterozygous markers; real-time gating hardware configured to receive the optical vector and to generate a sorting decision based on the optical vector; an indexed collection module configured to receive the labeled sperm at an indexed collection location based on the sorting decision, wherein the indexed collection location preserves an association between the labeled sperm and a digital record including the optical vector; and a processor configured to perform operations. The operations may include obtaining a primary aliquot of a live sperm sample; incubating the primary aliquot with an allele-discriminating reagent panel to produce a labeled sperm, wherein the allele-discriminating reagent panel comprises a plurality of allele-discriminating reagents respectively directed to heterozygous markers selected based on phased paternal genomic information, a male recombination map, and one or more constraints; detecting an optical signal from the labeled sperm using an optical interrogation instrument; determining an optical vector based on the optical signal; sorting the labeled sperm to an indexed collection location, wherein the indexed collection location preserves an association between the labeled sperm and a digital record including the optical vector; determining a posterior probability distribution for the labeled sperm based on the optical vector and one or more prior inputs; and determining a genome-wide statistical trait prediction based on the posterior probability distribution.

In some aspects, the optical interrogation instrument comprises a multi-color flow cytometer or an optical-imaging flow cytometer. In some aspects, the real-time gating hardware comprises a field-programmable gate array. In some aspects, the real-time gating hardware is configured to generate the sorting decision within less than or equal to 100 microseconds. In some aspects, the indexed collection module comprises a micro-well plate, a nano-well array, a droplet format, a barcoded droplet format, or a dielectrophoretic array. In some aspects, the posterior probability distribution is configured to use a Hidden Markov Model or an equivalent probabilistic graphical model. In some aspects, the one or more prior inputs further comprise channel-specific error parameters and accessibility parameters. In some aspects, the set of heterozygous markers are selected based on the phased paternal genomic information, the male recombination map, and one or more constraints.

In some aspects, the system may further include a genome-wide prediction engine configured to derive an expected allele dosage at a plurality of loci and at least one genome-wide statistical trait prediction based on the posterior probability distribution. In some aspects, the system may further include a utility-ranking module configured to rank a plurality of labeled sperm according to a utility function based on respective genome-wide statistical trait predictions.

Further provided herein are non-transitory computer readable media storing instructions that, when executed by a processor, cause the processor to perform operations comprising the steps of any method disclosed herein.

The foregoing general description of the illustrative embodiments and the following detailed description thereof are merely exemplary aspects of the teachings of this disclosure and are not restrictive.

### BRIEF DESCRIPTION OF DRAWINGS

Non-limiting and non-exhaustive examples are described with reference to the following figures.

FIG. 1 illustrates a schematic workflow for probabilistic single-sperm genome reconstruction and selection, according to aspects of the present disclosure.

FIG. 2 illustrates a flowchart for a method for determining a genome-wide statistical trait prediction from a sperm cell, according to aspects of the present disclosure.

FIG. 3 illustrates a flowchart for a method for producing an allele-discriminating reagent panel for a sperm cell, according to aspects of the present disclosure.

FIG. 4 illustrates a schematic diagram of a panel design module configured to generate a selected panel specification, according to aspects of the present disclosure.

FIG. 5A illustrates an exemplary allele-discriminating reagent with direct signal generation, according to aspects of the present disclosure.

FIG. 5B illustrates an exemplary allele-discriminating reagent with indirect signal generation, according to aspects of the present disclosure.

FIG. 6 illustrates a schematic diagram of an optical interrogation system for measuring optical signals from labeled sperm, according to aspects of the present disclosure.

FIG. 7 illustrates a flowchart for a method for determining a sorting decision based on an optical vector, according to aspects of the present disclosure.

FIG. 8 illustrates exemplary indexed collection formats for sorted sperm, according to aspects of the present disclosure.

FIG. 9 illustrates a schematic diagram of a probabilistic reconstruction framework for determining posterior haplotype probabilities, according to aspects of the present disclosure.

FIG. 10 illustrates a flowchart for a method for determining a genome-wide prediction for a labeled sperm, according to aspects of the present disclosure.

FIG. 11 illustrates a block diagram of a computing environment for probabilistic single-sperm genome reconstruction and selection, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

### Definitions

As used herein, the term “sperm” may refer to a sperm cell, spermatozoon, or other male gamete, and may include sperm obtained from a fresh ejaculate, processed sample, washed sample, cryopreserved sample, thawed sample, enriched sample, or other preparation suitable for assisted reproductive use.

As used herein, the term “live sperm” refers to sperm that remain intact and are not intentionally fixed prior to optical interrogation, and may include sperm that retain full viability, partial viability, motility, fertilization potential, or a combination thereof sufficient for a contemplated downstream use.

As used herein, the term “marker” refers to a genomic feature used for inference regarding haplotype origin, recombination structure, genotype state, or predicted trait value. In many embodiments, the marker is a heterozygous single-nucleotide polymorphism in a paternal genome. In other embodiments, additional or alternative polymorphic loci may be used.

As used herein, the term “selected heterozygous marker” refers to a paternal heterozygous locus chosen for use in a measurement panel based on an optimization criterion related to haplotype information content, genome coverage, recombination-aware spacing, assay performance, fluorescence-channel availability, expected chromatin accessibility, or a combination thereof.

As used herein, the term “allele-discriminating reagent” refers to any probe, probe set, opener, reporter-binding reagent, or other molecule or reagent set configured to distinguish between alternative alleles or haplotype states at a selected locus directly or indirectly. An allele-discriminating reagent may itself be fluorescent, may recruit one or more secondary reporters, may create a structural opening or binding substrate for a secondary reporter, or may serve as a substrate for localized signal amplification.

As used herein, the terms “label”, “tag”, “labeling”, and “tagging” refer to the association of an allele-discriminating reagent or reporter with a sperm cell such that the sperm cell produces a detectable optical signal corresponding to one or more targeted loci. A labeled or tagged sperm is a sperm that has been incubated with an allele-discriminating reagent panel and is capable of generating a genotype-associated optical measurement. The terms may be used interchangeably throughout this disclosure.

As used herein, the term “paternal haplotypes” refers to phased paternal genomic sequences or phased paternal variant states, which may be represented as a first paternal haplotype and a second paternal haplotype, such as H(0) and H(1), for purposes of downstream probabilistic inference.

As used herein, the term “male recombination map” or “sex-specific recombination map” refers to information characterizing expected crossover rates, recombination frequencies, genetic distances, or related recombination structure along one or more chromosomes for male meiosis.

As used herein, the term “accessibility prior” refers to any computationally derived, empirically measured, or externally provided estimate of the relative likelihood that a selected locus will yield a measurable signal in sperm chromatin. Accessibility priors may be derived from chromatin-state annotations, retained-nucleosome maps, GC content, CpG density, sequence context, donor-characterized calibration data, or combinations thereof.

As used herein, the term “genotype-associated optical measurement” refers to any optical measurement associated with an individual sperm that conveys information regarding one or more targeted loci, haplotypes, or genotype states. A genotype-associated optical measurement may include direct fluorescence from an allele-discriminating reagent, fluorescence or other optical signal generated by a secondary reporter, fluorescence or other optical signal generated by a localized amplification product, binary values, ternary values, continuous intensities, spectrally unmixed values, normalized values, transformed values, confidence measures, or any combination thereof across one or more channels or derived features.

As used herein, the term “real-time processing hardware” refers to circuitry, logic, processors, or combinations thereof configured to perform a gating or control computation on a timescale compatible with optical sorting of individual sperm. Real-time processing hardware may include, for example, a field-programmable gate array, application-specific integrated circuitry, digital-signal-processing circuitry, a graphics processor, a central processing unit, or combinations thereof.

As used herein, the term “accepted sperm” refers to a sperm that satisfies a sorting criterion, threshold, classification, target-pattern match, score condition, or other decision rule and is therefore retained, diverted, recorded, or otherwise designated for downstream handling.

As used herein, the term “indexed collection location” refers to a physical location to which a sperm is directed, retained, or associated, where the location has an address, identifier, barcode, coordinate, position, or other index preserving a link between the physical sperm and corresponding digital information. Exemplary indexed collection locations include wells, micro-wells, nano-wells, droplets, barcoded droplets, and dielectrophoretic locations.

As used herein, the term “posterior probability distribution” refers to a probability distribution generated after considering observed data, such as one or more optical measurements, and may describe uncertainty over haplotype states, locus states, crossover positions, genotype states, or combinations thereof.

As used herein, the term “expected allele dosage” refers to an estimate, which may be probabilistic, of allele content at one or more loci, and may be derived from a posterior distribution over paternal haplotypes, genotype states, or related hidden variables.

As used herein, the term “statistical trait predictor” refers to any quantitative or categorical predictor derived from genomic information, including a polygenic score, polygenic index, risk score, liability estimate, trait estimate, disease-risk estimate, breeding-value estimate, or other genome-informed prediction.

As used herein, the term “utility function” refers to a scoring, ranking, filtering, or decision rule based on one or more inputs, such as one or more statistical trait predictors, uncertainty terms, monogenic exclusion criteria, chromosome-state criteria, sex-chromosome criteria, quality-control outputs, or combinations thereof. A utility function may be scalar, vector-valued, threshold-based, rule-based, lexicographic, or otherwise structured.

As used herein, the term “assisted reproductive technology” or “ART” includes intracytoplasmic sperm injection, in vitro fertilization, intrauterine insemination, cryopreservation workflows, embryo-generation workflows, embryo-testing workflows, veterinary breeding workflows, and other reproductive procedures involving sperm selection or handling.

### Overview of System Architecture

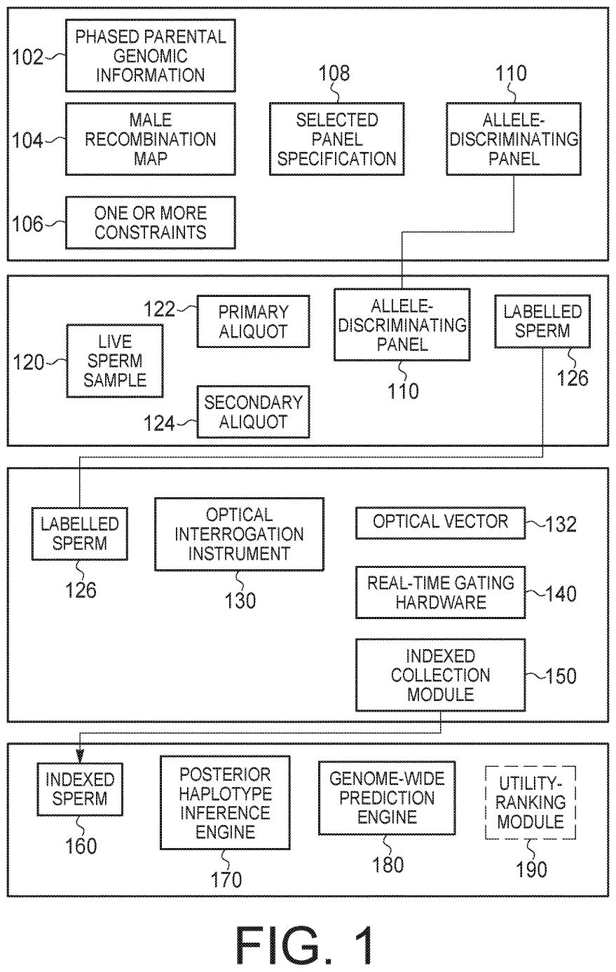

FIG. 1 illustrates a system architecture 100 for probabilistic single-sperm genome reconstruction and selection. The system architecture 100 may be used in human assisted reproduction or in veterinary breeding or other non-human reproductive settings. The system architecture 100 provides an integrated platform for selecting sperm prior to fertilization based on probabilistic inference of paternal genetic content from sparse, donor-specific marker measurements.

The system architecture 100 receives multiple inputs that feed into the generation of reagents for sperm labeling. Phased paternal genomic information 102 may identify heterozygous loci across the genome and may define a first paternal haplotype and a second paternal haplotype. A male recombination map 104 may specify genetic distance, crossover rate, local recombination rate, recombination probability, or related recombination information as a function of genomic position. One or more constraints 106 may include chromatin-accessibility priors, fluorescence-channel constraints, recombination-based spacing constraints, or combinations thereof. The phased paternal genomic information 102, the male recombination map 104, and the one or more constraints 106 feed into the generation of a selected panel specification 108.

The selected panel specification 108 may include marker identities, chromosomal locations, paternal allele assignments, reagent identities, fluorophore identities, channel mappings, accessibility estimates, or combinations thereof. Based on the selected panel specification 108, an allele-discriminating reagent panel 110 may be produced. The allele-discriminating reagent panel 110 may comprise a plurality of allele-discriminating reagents respectively directed to heterozygous markers selected based on the phased paternal genomic information 102 and the male recombination map 104. In some cases, the allele-discriminating reagent panel 110 may comprise allele-discriminating reagents targeted to heterozygous single-nucleotide polymorphisms selected from a paternal genome based on haplotype information content under the male recombination map 104.

A live sperm sample 120 may be divided into a primary aliquot 122 and a secondary aliquot 124. The primary aliquot 122 may be combined with the allele-discriminating reagent panel 110 to produce labeled sperm 126. In some cases, the labeled sperm 126 may be produced by incubating a sperm with the allele-discriminating reagent panel 110. The secondary aliquot 124 may be used for calibration or refinement purposes in connection with the allele-discriminating reagent panel 110. In some cases, data from the secondary aliquot 124 may be used to estimate locus-specific accessibility, signal intensity, background, hybridization success, amplification success, channel-specific error behavior, or combinations thereof.

The labeled sperm 126 may be processed by an optical interrogation instrument 130. The optical interrogation instrument 130 may be configured to detect an optical signal from the labeled sperm 126 and to generate an optical vector 132 based on the optical signal. In some cases, the optical interrogation instrument 130 may comprise a multi-color flow cytometer or an optical-imaging flow cytometer. In some cases, the optical interrogation instrument 130 may be configured to generate an optical vector 132 for each sperm in a population of live sperm. The optical interrogation instrument 130 may include multiple excitation sources and multiple detection channels sufficient to resolve selected fluorophores or other reporter outputs. In some cases, the optical interrogation instrument 130 may comprise at least 30 spectral channels. In some cases, the optical interrogation instrument 130 may comprise four lasers and about 30 to about 50 detection channels capable of spectral unmixing.

The optical vector 132 may be evaluated by real-time gating hardware 140. The real-time gating hardware 140 may be configured to receive the optical vector 132 and to generate a sorting decision based on the optical vector 132. In some cases, the real-time gating hardware 140 may comprise real-time processing hardware or circuitry configured to compute a gating score from the optical vector 132 or to match the optical vector 132 to a stored target pattern, and to generate a sorting control signal. In some cases, the real-time gating hardware 140 may comprise a field-programmable gate array. In some cases, the real-time gating hardware 140 may be configured to generate the sorting decision within less than 100 microseconds.

Sperm satisfying the sorting criteria may be directed to an indexed collection module 150. The indexed collection module 150 may be configured to receive the labeled sperm 126 at an indexed collection location based on the sorting decision. The indexed collection location may preserve an association between the labeled sperm 126 and a digital record including the optical vector 132. In some cases, the indexed collection module 150 may be configured to receive individually sorted sperm in indexed collection locations. The indexed collection module 150 may comprise a micro-well plate, a nano-well array, a droplet generator, a barcoded droplet format, or a dielectrophoretic array.

For each sorted sperm, a posterior haplotype inference engine 160 may determine a posterior probability distribution over paternal haplotypes. The posterior haplotype inference engine 160 may be configured to determine the posterior probability distribution for the labeled sperm 126 based on the optical vector 132 and one or more prior inputs comprising paternal haplotype information and the male recombination map 104. In some cases, the posterior haplotype inference engine 160 may use a probabilistic graphical model that incorporates the paternal haplotype information, the male recombination map 104, and channel-specific error parameters. In some cases, the posterior haplotype inference engine 160 may use a Hidden Markov Model or an equivalent probabilistic graphical model.

The output from the posterior haplotype inference engine 160 may feed into a genome-wide prediction engine 170. The genome-wide prediction engine 170 may derive an expected allele dosage at a plurality of loci and may calculate at least one genome-wide statistical trait predictor based on the posterior probability distribution. In some cases, the genome-wide prediction engine 170 may derive at least one genome-wide statistical trait predictor with a confidence interval. The genome-wide statistical trait predictor may include a polygenic score, a disease-risk estimate, a liability estimate, a breeding-value estimate, or a combination thereof.

A utility-ranking module 180 may then apply a utility function to rank or filter sperm based on predicted traits, uncertainty measures, and optional exclusion criteria. In some cases, the utility-ranking module 180 may be configured to rank a plurality of labeled sperm according to a utility function based on respective genome-wide statistical trait predictors. In some cases, the utility-ranking module 180 may select at least one sorted sperm for fertilization according to the utility function. The utility function may consider one or more predicted traits, disease-risk estimates, confidence measures, or crossover-related quality-control information. In some cases, the utility function may exclude sperm predicted to carry a specified monogenic risk allele or a specified sex-chromosome state.

The process may conclude with the identification of selected sperm 190 for downstream assisted reproductive technology handling. The selected sperm 190 may be used directly in an assisted reproductive procedure, such as intracytoplasmic sperm injection. In some cases, the sperm-stage analysis may be followed by embryo generation and embryo-level testing, and sperm-stage posterior information may be combined with embryo-genotype information to generate a refined embryo ranking.

### Allele-Discriminating Reagent Panels

The present disclosure provides for the design and preparation of allele-discriminating reagent panels, which may be used to label or tag sperm cells for various downstream applications. As discussed in further detail below, paternal genomic input data and preprocessing may be used for marker-panel design and preparation.

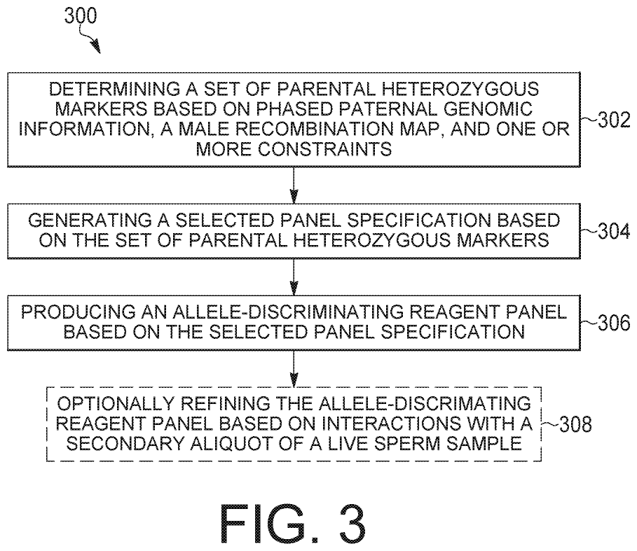

FIG. 3 illustrates an exemplary method 300 for designing and preparing an allele-discriminating reagent. The method 300 may begin with the step 302 of determining a set of parental heterozygous markers based on phased paternal genomic information 102, a male recombination map 104, and one or more constraints 106. Step 302 may include preprocessing steps of inputs followed by determination of the set of parental heterozygous markers. In some embodiments, a preprocessing module may perform the preprocessing steps by preparing the phased paternal genomic information, a male recombination map, and one or more constraints in the form of a donor-specific design package, and providing the design package to a panel design module 400 for the determination step. The design package may include a selected list of candidate loci, paternal haplotype information for those loci, fluorophore-allocation constraints, expected recombination-aware informativeness, accessibility priors, assay notes, quality filters, trait-model links, utility settings, or combinations thereof. This design package may be used to drive reagent synthesis, reagent selection, gating configuration, and later probabilistic inference. In other embodiments, the panel design module 400 may receive the phased paternal genomic information, a male recombination map, and one or more constraints and perform the preprocessing steps and determination step.

Phased paternal genomic information 102 includes paternal genomic input data sufficient to characterize heterozygous loci and phased haplotype structure. Such input data may include phased whole-genome sequence data for a sperm donor or other paternal subject. Phased variant data may be generated by sequencing, imputation, family-based phasing, population-based phasing, long-read phasing, linked-read phasing, statistical phasing, or combinations thereof. The phased input may be represented as two paternal haplotypes defined across autosomes and, where relevant, sex chromosomes. In some embodiments, a preprocessing module may be involved in producing the phased paternal genomic information. The preprocessing module may identify loci suitable for downstream interrogation. In some embodiments, candidate loci are filtered to retain paternal heterozygous loci. Optionally, candidate loci may additionally be filtered according to one or more assay-oriented criteria, such as probe-design suitability, local sequence context, uniqueness of hybridization target, absence of problematic nearby variants, fluorescence-panel compatibility, chromosomal distribution, expected information gain, accessibility priors, or combinations thereof.

A male recombination map 104 may specify genetic distance, crossover rate, local recombination rate, recombination probability, or related recombination information as a function of genomic position. The male recombination map may be a genome-wide recombination map, a chromosome-specific or region-specific recombination map, a population-derived map, or a customized map (e.g., a donor-adjusted, ancestry-adjusted, or otherwise customized). In some embodiments, one or more genome-wide predictor models may produce the male recombination map. The one or more genome-wide predictor models may include effect weights, scoring coefficients, locus sets, transformation rules, or post-processing rules associated with one or more target traits, disease risks, or selection goals. In some embodiments, the predictor models are not used to choose markers directly, but are used later to define or evaluate a utility function after posterior inference. In some embodiments, predictor-model information may nevertheless be used as one input to a panel-design procedure, such as by weighting information content according to clinical relevance or expected downstream decision value.

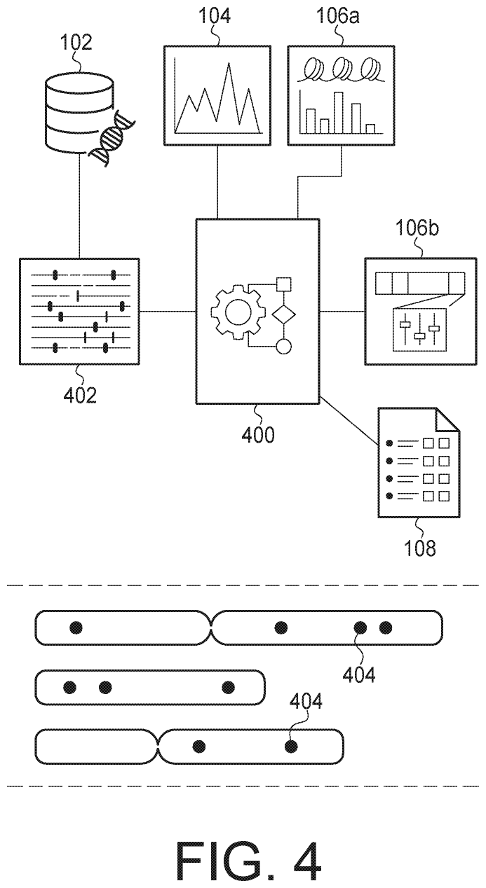

The one or more constraints 106 may include a chromatin-accessibility prior, a fluorescence-channel constraint, a recombination-based spacing constraint, or a combination thereof. For example, as illustrated in FIG. 4, the one or more constraints 106 may include a chromatin-accessibility prior 106a and a fluorescence-channel constraint 106b. The chromatin-accesibility prior 106a may be produced as part of preprocessing ingestion of published or donor-characterized retained-nucleosome maps, ChIP-seq tracks, MNase-seq tracks, ATAC-seq tracks, GC-content profiles, CpG-density profiles, promoter annotations, imprinted-locus annotations, HOX-cluster annotations, or other genomic annotations associated with expected accessibility of selected loci in sperm chromatin. The chromatin-accessibility prior 106a may be derived from retained-nucleosome annotations, promoter annotations, CpG density, GC content, imprinted-locus annotations, HOX-cluster annotations, donor-characterized chromatin-state maps, or combinations thereof. In some cases, the chromatin-accessibility prior 106a may be derived from retained-nucleosome domains, GC-rich promoter regions, imprinted gene clusters, HOX clusters, CpG-rich regions, sequence-derived accessibility priors, or combinations thereof. Candidate loci may be filtered or prioritized according to overlap with retained-nucleosome domains, CpG-rich promoter regions, imprinted gene clusters, HOX clusters, GC-rich regions, other chromatin-state annotations, or other loci predicted or observed to be more accessible than heavily compacted protamine-dominated regions. In some cases, accessibility-aware filtering may be used to increase the probability that the resulting panel contains loci that are not only informative in recombination space but also capable of yielding measurable optical signal in live sperm. In some cases, accessibility-aware filtering may be performed computationally before reagent ordering. Published or donor-derived chromatin-state maps may be intersected with the donor's heterozygous single-nucleotide polymorphism set and the male recombination map 104 to identify loci that satisfy both informativeness and accessibility criteria. In some cases, sequence-derived features, such as fine-scale GC-content variation, CpG density, or promoter annotations, may be used as accessibility priors when direct donor-specific chromatin maps are unavailable. The fluorescence-channel constraint 106b may correspond to a number of available fluorescence channels. The number of available fluorescence channels may correspond to a desired marker-panel size or to a number of distinct probe states or derived features that can be reliably discriminated by an optical system. In some cases, the panel design module 400 may target a panel containing at least 10 markers, at least 20 markers, at least 30 markers, about 20 to about 40 markers, or another number suitable for a given instrument and workflow.

The one or more constraints 106 may further include user-specific or clinic-specific configuration. By way of example, a user, physician, laboratory, embryologist, breeder, or other operator may specify one or more desired traits, undesired outcomes, exclusionary alleles, chromosome-state filters, confidence thresholds, throughput targets, maximum panel sizes, instrument constraints, cost constraints, or combinations thereof. These parameters may be stored and later used during marker selection, gating, ranking, or downstream embryo integration.

As illustrated in FIG. 4, preprocessing steps may include processing the phased paternal genomic information 102 to identify heterozygous candidate loci 402. The heterozygous candidate loci 402 may represent paternal heterozygous single-nucleotide polymorphisms or other polymorphic loci suitable for downstream interrogation. In some cases, the heterozygous candidate loci 402 may be filtered according to one or more assay-oriented criteria, such as probe-design suitability, local sequence context, uniqueness of hybridization target, absence of problematic nearby variants, fluorescence-panel compatibility, chromosomal distribution, expected information gain, accessibility priors, or combinations thereof. Panel design module 400 may select markers from the heterozygous candidate loci 402 for interrogation at target locus 404 positions. In some cases, panel design module 400 may use a greedy sub-modular maximization approach that iteratively adds the candidate locus providing the largest marginal gain in a selected information criterion. The selected information criterion may include expected reduction in uncertainty regarding haplotype origin, expected variance explained in one or more downstream predictors, expected discrimination among candidate sperm, expected posterior sharpness, expected utility gain, or combinations thereof.

The panel design module 400 may penalize selection of markers closer than a specified genetic distance under the male recombination map 104 to ensure broad chromosomal coverage. Markers that are too close together in genetic distance may provide redundant information because the markers are unlikely to be separated by recombination in a single meiosis. In some cases, the optimization procedure may incorporate spacing based on the recombination map by penalizing selection of markers closer than a specified genetic distance, for example, using a term proportional to e^{-2d}ₙₑₐᵣₑₛₜ^{(s, S)}, where d is genetic distance in Morgans. In some cases, the optimization procedure may require minimum spacing between markers, require coverage across different chromosome arms, require representation across multiple chromosomes, or otherwise account for recombination structure in allocating a limited number of assay channels.

The set of parental heterozygous markers may be selected to maximize haplotype information content across chromosomes under the male recombination map. In some cases, the set of parental heterozygous markers may be selected to provide broad and informative resolution of paternal haplotype state so that genome-wide allele dosage and downstream trait predictors can be inferred probabilistically from sparse measurements. In some cases, determining the set of parental heterozygous markers may comprise iteratively selecting single-nucleotide polymorphisms according to marginal information gain while penalizing single-nucleotide polymorphisms closer than a specified genetic distance under the male recombination map. Markers that are too close together in genetic distance may provide redundant information because the markers are unlikely to be separated by recombination in a single meiosis.

In some cases, the set of parental heterozygous markers may be independent of an effect-allele set used to calculate a downstream statistical trait predictor. The independence may be complete or partial. The marker set may include loci chosen primarily for haplotype informativeness rather than direct predictive effect size, while the downstream predictor may be computed using expected allele dosage across a much larger set of loci, including loci not directly interrogated by the measurement panel. In this manner, sparse measurements at selected loci may support probabilistic inference of broader genome-wide genetic information.

Returning to FIG. 3, the method 300 may further include a step 304 of generating a selected panel specification 108 based on the set of parental heterozygous markers. Step 304 may be performed by panel design module 400, as illustrated in FIG. 4. The selected panel specification 108 may include marker identities, chromosomal locations, paternal allele assignments, reagent identities, fluorophore identities, channel mappings, accessibility estimates, or combinations thereof. In some cases, the selected panel specification may further include predicted informativeness, expected recombination-aware spacing characteristics, reporter identities, concentrations, hybridization notes, or quality thresholds. The selected panel specification 108 may be stored as a donor-specific file, transmitted to a synthesis or selection module, loaded into a sorter-control system, or associated with later inference software.

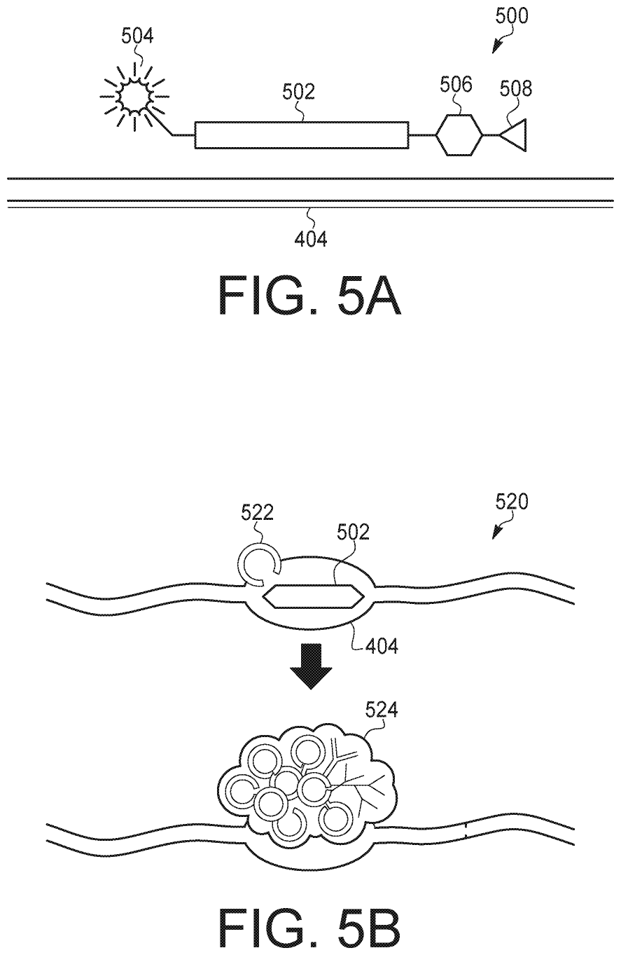

Returning to FIG. 3, the method 300 may further include a step 306 of producing an allele-discriminating reagent panel 110 based on the selection panel specification 108. The selected marker panel specification 108 may be implemented using a plurality of allele-discriminating reagents configured to interrogate donor-specific heterozygous loci in live sperm. The reagents (e.g., allele-discriminating reagents) may be designed after selection of a donor-specific marker set as described above, such that each reagent corresponds to a selected heterozygous locus and is associated with a direct or indirect optical signal. In some embodiments, the plurality of reagents is individualized to a particular donor and is selected to support genome-wide haplotype inference from sparse measurements rather than direct measurement of every locus later used in a downstream statistical trait predictor. In some embodiments, the reagents are configured to discriminate between alternative alleles at a selected heterozygous SNP and to generate, recruit, or enable distinguishable fluorescence outputs when associated with tagged sperm are processed by a multi-color cytometer. In some cases, the allele-discriminating reagent panel 110 may comprise peptide nucleic acid (PNA) oligomers, locked nucleic acid (LNA) oligomers, gamma-PNA oligomers, bis-PNA oligomers, or combinations thereof as nucleic-acid analog probes. In some cases, the allele-discriminating reagent panel 110 may comprise about 20 to about 40 allele-discriminating reagents. The allele-discriminating reagents may be labeled with distinct fluorophores resolvable without greater than about 5% spectral overlap. The allele-discriminating discriminating reagents may be provided according to various configurations, such as the exemplary configurations illustrated in FIG. 5A and FIG. 5B.

FIG. 5A illustrates an allele-discriminating reagent 500 with a direct signal-generation configuration. The allele-discriminating reagent 500 may include a target-binding region 502 configured to bind to a target locus 404. The target-binding region 502 may be configured to engage the target locus 404 in an allele-discriminating manner such that the allele-discriminating reagent 500 binds preferentially to one allele over an alternative allele at a heterozygous site. In some cases, the target-binding region 502 may comprise about 15 to about 20 nucleotides or nucleotide analog positions in length for improved allele discrimination and binding specificity. The allele-discriminating reagent 500 may include a fluorophore 504 for direct optical signal generation, such as Cy5 or Alexa Fluor dyes. The fluorophore 504 may be attached to the target-binding region 502. In some cases, the fluorophore 504 may be conjugated to the allele-discriminating reagent 500 via a terminal lysine or comparable reactive moiety linked to a maleimide-activated dye. In some cases, the fluorophore 504 may be selected to be resolvable by a chosen optical instrument, such as a multi-channel cytometer. The allele-discriminating reagent 500 may further comprise an uptake motif 506 to facilitate cellular entry. In some cases, the uptake motif 506 may comprise a cellular-uptake motif such as a cell-penetrating peptide. In some cases, the cell-penetrating peptide may include Tat, Penetratin, or comparable motifs compatible with live-sperm handling. The allele-discriminating reagent 500 may further comprise an optional localization motif 508 positioned to promote nuclear access. In some cases, the allele-discriminating reagent 500 may comprise a cellular-uptake motif, a nuclear-localization motif, or a combination thereof.

FIG. 5B illustrates an allele-discriminating reagent 520 with an indirect signal-generation configuration. The allele-discriminating reagent 520 may engage the target locus 404 through a target-binding region 502. In the indirect signal-generation configuration, the allele-discriminating reagent 520 may not itself be the primary fluorescent signal source. Rather, the allele-discriminating reagent 520 may be configured to form a localized structure at the target locus 404. The localized structure may create a structural opening, local hybridization substrate, reporter-binding site, ligatable configuration, or other detectable state that is subsequently converted into a localized fluorescent signal. The localized structure may be created or formed through the use of an opener reagent. A secondary reporter 522 may be associated with the allele-discriminating reagent 520. The localized structure may be configured to bind the secondary reporter 522 capable of generating a fluorescent signal. The secondary reporter 522 may bind, ligate, extend, amplify, decorate, or otherwise generate a localized fluorescent signal associated with the allele-discriminating event at the target locus 404. A localized amplification product 524 may be generated from the target locus 404, resulting in a bright optical signal that may be measured by an optical interrogation instrument. In some cases, the secondary reporter 522 or the localized amplification product 524 may be generated using padlock probes, rolling circle amplification, hybridization chain reaction, branched DNA amplification, enzymatic reporters, or combinations thereof. In some cases, localized signal amplification may be used when direct single-reagent fluorescence would otherwise be below a desired sorter detection threshold. The localized amplification product 524 may produce a spatially localized multi-copy fluorescent output from a single underlying allele-discriminating event.

In some cases, the allele-discriminating reagent panel 110 may include at least one reagent targeting a Y-chromosome locus. The Y-chromosome-targeting reagent may be used in workflows incorporating sex-chromosome filtering or ranking criteria.

Returning to FIG. 3, the method 300 may optionally further include a step 308 of refining the allele-discriminating reagent panel 110 based on interactions with a secondary aliquot of a live sperm sample. An allele-discriminating reagent panel 110 may be designed for use with a particular live sperm sample, representing a sample of sperm cells obtained from fresh ejaculate and prepared for live-cell interrogation. In some embodiments, the live sperm sample is not intentionally fixed, but rather, is compatible with live-sperm handling. The live sperm sample may be separated into a primary aliquot and a secondary aliquot. The secondary aliquot may be used for refining the allele-discriminating reagent panel 110. In other words, the secondary aliquot may represent a “sacrificial aliquot” or a “calibration aliquot” that includes sperm cells that will not be indexed or preserved. The secondary aliquot may be contacted with a candidate panel or subsets of reagents and may be used to estimate locus-specific accessibility, signal intensity, background, hybridization success, amplification success, channel-specific error behavior, or combinations thereof. Data from the calibration aliquot may be used to refine reagent concentrations, incubation conditions, gating thresholds, panel composition, emission probabilities, accessibility parameters, or all of the foregoing before the primary sperm population is processed.

### Genome-Wide Predictions of Live Sperm Cells

The allele-discriminating reagent panels of the present disclosure may be used in methods and systems for determining genome-wide statistical trait predictions from live sperm cells. These methods and systems may involve preparing live sperm samples, labeling the live sperm samples with the allele-discriminating reagent panels, optically interrogating a labeled sperm cell from the live sperm samples, and using the optical data to predict traits for the labeled sperm cell. The labeled sperm cell may then be sorted, preserved, and stored in an indexed collection location that maintains an association between the location of the stored labeled sperm cell and a digital record that includes the predicted traits, data, and metadata for the stored labeled sperm cell. These method and systems may be used for various downstream applications, such as human assisted reproduction or in veterinary breeding or other non-human reproductive settings, and pre-fertilization genetic assessment by converting sparse optical measurements into broader genomic predictions through probabilistic inference.

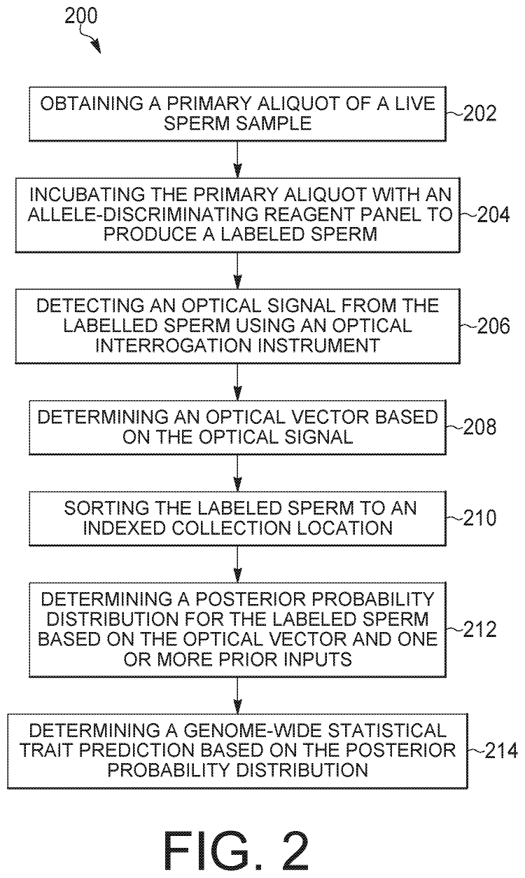

FIG. 2 illustrates an exemplary method 200 for determining a genome-wide statistical trait prediction from a sperm cell. The method 200 may begin with a step 202 of obtaining a primary aliquot 122 of a live sperm sample, that includes a population of live sperm cells suitable for downstream labeling and optical interrogation. The live sperm sample may be obtained from a fresh ejaculate or processed sperm sample that is prepared for live-cell interrogation and contacted with a plurality of allele-discriminating reagents under conditions selected to preserve downstream usability of at least a subset of the sperm population. In some embodiments, the live sperm sample is not intentionally fixed before optical interrogation. Rather, the disclosed workflows contemplate live-sperm handling compatible with subsequent sorting, indexed retention, retrieval, fertilization, or cryopreservation. The live sperm sample may be separated into a primary aliquot 122 and a secondary aliquot 124. The primary aliquot 122 may be used for step 202 of method 200. The secondary aliquot 124 may be processed by a small test module for calibration of gating parameters used by real-time gating hardware, and for refinement of an allele-discriminating reagent panel as described with respect to step 308 of method 300.

The method 200 may further include the step 204 of incubating the primary aliquot 122 with an allele-discriminating reagent panel 110 to produce a labeled sperm 126. The incubation may be performed under conditions that preserve sperm viability while permitting the allele-discriminating reagents to engage target loci within the sperm. In some embodiments, a sperm sample is incubated with a pool of reagents, such as a donor-specific panel containing m reagents, each directed to a selected heterozygous locus. By way of example, reagent concentrations may be on the order of about 1 micromolar per reagent, and the incubation may be performed in a suitable buffer, for example a buffer containing about 150 mM NaCl, at a temperature in a range of about 25 degrees C. to about 37 degrees C. for a duration of about 30 minutes. These values are illustrative, and in some embodiments one or more conditions may be adjusted to account for sample source, reagent composition, instrument schedule, or desired balance between signal strength and downstream sperm performance. In some embodiments, incubation conditions are selected to preserve motility and viability relative to appropriate controls. For example, conditions may be adjusted such that sperm motility remains at least about 75% of a control sample after reagent exposure. In some embodiments, the workflow may include optional washing, dilution, buffer exchange, or transfer steps after incubation and before optical interrogation. In some embodiments, one or more secondary reporters or localized amplification reagents are introduced after initial allele-discriminating binding and before optical interrogation. In some embodiments, the tagged or otherwise optically marked sample may be handled as a bulk population until interrogation, while in other embodiments pre-sort enrichment, debris filtering, or other sample-conditioning operations may be performed. In each case, the purpose of the tagging stage is to convert donor-specific genomic design information into an information-rich optical representation that can be measured at the level of individual live sperm and subsequently interpreted using the inference framework described below.

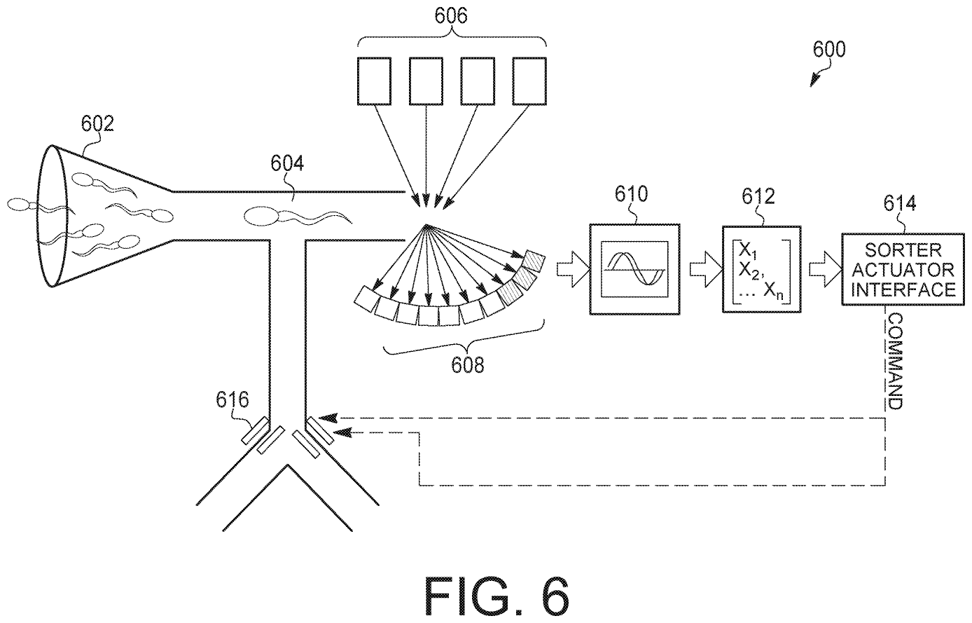

The method 200 may further include a step 206 of detecting an optical signal from the labeled sperm 126 using an optical interrogation instrument 130. The optical interrogation instrument 130 may comprise a multi-color flow cytometer or an optical-imaging flow cytometer, as described above with reference to FIG. 1. In some cases, the optical interrogation instrument 130 may comprise at least 30 spectral channels. The optical signal may comprise fluorescence emissions, spectral signatures, or other genotype-associated optical outputs generated by allele-discriminating reagents or by secondary reporters associated with allele-discriminating reagents. In some embodiments, optical interrogation instrument 130 may correspond to or be part of an exemplary optical interrogation system 600 as illustrated by FIG. 6.

As illustrated in FIG. 6, the optical interrogation system 600 may include a sample inlet 602 through which a sperm sample enters the optical interrogation system 600. The sample inlet 602 may be configured to receive labeled sperm from an upstream incubation or preparation stage. The labeled sperm may pass from the sample inlet 602 to an interrogation region 604. The interrogation region 604 may be configured to receive individual sperm for optical measurement. One or more excitation sources 606 may be positioned to direct light toward the interrogation region 604. The excitation sources 606 may illuminate labeled sperm as the tagged sperm pass through the interrogation region 604. In some cases, the excitation sources 606 may comprise multiple lasers configured to excite different fluorophores or reporter outputs associated with the allele-discriminating reagent panel. Detection channels 608 may be arranged to capture fluorescence signals across multiple spectral ranges. The detection channels 608 may collect optical emissions from the labeled sperm resulting from illumination by the excitation sources 606. In some cases, the detection channels 608 may be arranged in an array configuration to capture fluorescence signals across a plurality of spectral ranges corresponding to the fluorophores used in the allele-discriminating reagent panel. The optical interrogation system 600 may comprise a multi-color flow cytometer or an optical-imaging flow cytometer. In some cases, the multi-color flow cytometer may comprise at least 30 spectral channels. In some cases, the multi-color flow cytometer may comprise four lasers and about 30 to about 50 detectors capable of spectral unmixing. The number of effectively resolvable channels may be selected to match or exceed the number of markers included in a given donor-specific panel. In some cases, the optical interrogation system 600 may operate at a throughput of at least about 30,000 events per second. The throughput may permit large sperm populations to be interrogated within clinically practical time windows.

Returning to FIG. 2, the method 200 may further include a step 208 of determining an optical vector based on the optical signal. The optical vector may represent a multi-dimensional measurement corresponding to the detected optical signal. In some cases, the optical vector may be generated by spectral unmixing or other signal-processing operations applied to the raw optical signal. The optical vector may encode information about allele presence or absence at the heterozygous markers targeted by the allele-discriminating reagent panel. As illustrated in FIG. 6, the signals from the detection channels 608 may be processed by a spectral-unmixing block 610. The spectral-unmixing block 610 may separate overlapping fluorescence signals to generate an optical vector output 612. The spectral-unmixing block 610 may apply spectral unmixing algorithms to resolve contributions from different fluorophores that may have partially overlapping emission spectra. The optical vector output 612 may represent processed fluorescence data associated with each sperm. In some cases, the optical vector output 612 may be represented as a binarized vector indicating allele presence or absence at interrogated loci. In some cases, the optical vector output 612 may be represented as a ternary vector indicating on, off, or ambiguous states. In some cases, the optical vector output 612 may be represented as a quantitative vector with continuous intensities. The representation used for gating may be chosen to balance speed, robustness, and downstream informativeness. In some cases, the optical interrogation system 600 may measure direct fluorescence from labeled allele-discriminating reagents. In some cases, the optical interrogation system 600 may measure fluorescence generated by secondary reporters or localized amplification products associated with an allele-discriminating event. In some cases, one or more channels may reflect direct reagent signal while one or more additional channels may reflect indirect or amplified signal, thereby allowing hybrid measurement architectures.

Returning to FIG. 2, the method 200 may further include a step 210 of sorting the labeled sperm to an indexed collection location. The indexed collection location may preserve an association between the labeled sperm and a digital record including the optical vector. In some cases, sorting the labeled sperm may comprise using real-time processing hardware to determine a gating computation for the labeled sperm, and the gating computation may be score-based or pattern-based. In some cases, the real-time processing hardware may comprise a field-programmable gate array. In some cases, the real-time processing hardware may generate the gating computation within less than 100 microseconds. The indexed collection location may comprise a micro-well plate, a nano-well array, a droplet or barcoded droplet format, or a dielectrophoretic array. The digital record may include the optical vector and may be stored in association with an identifier corresponding to the indexed collection location. As illustrated in FIG. 6, the optical vector output 612 may be supplied to a sorter actuator interface 614. The sorter actuator interface 614 may receive the optical vector output 612 and may generate control signals based on gating decisions. The sorter actuator interface 614 may interface with sorter timing, valve control, droplet charging, or equivalent actuation systems. A command signal from the sorter actuator interface 614 may be transmitted to deflection/droplet-charging plates 616. The deflection/droplet-charging plates 616 may direct sperm during sorting operations. The deflection/droplet-charging plates 616 may physically divert accepted sperm into indexed collection locations based on the sorting decisions generated from the optical measurements.

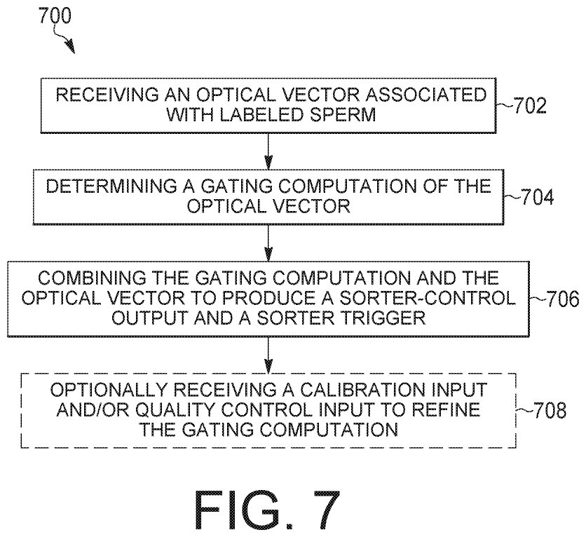

The step 210 of method 200 may include a step of determining a sorting decision based on the optical vector. FIG. 7 illustrates an exemplary method 700 for determining a sorting decision based on an optical vector. The method 700 may include the step 702 of receiving an optical vector associated with a labeled sperm. The optical vector may correspond to a set of heterozygous markers associated with an allele-discriminating panel. The optical vector may represent genotype-associated optical measurement data obtained from a labeled sperm that has been processed through an optical interrogation instrument. In some cases, the optical vector may be represented as a binarized vector indicating allele presence or absence at interrogated loci. In some cases, the optical vector may be represented as a ternary vector indicating on, off, or ambiguous states. In some cases, the optical vector may be represented as a quantitative vector with continuous intensities. The representation used for gating may be chosen to balance speed, robustness, and downstream informativeness.

The method 700 may further include a step 704 of determining a gating computation of the optical vector. In some cases, the gating computation may be score-based. In some cases, the gating computation may be pattern-based. The gating computation may be performed by real-time processing hardware. In some cases, the real-time processing hardware may comprise a field-programmable gate array. In some cases, the real-time processing hardware may comprise application-specific integrated circuitry, digital-signal-processing circuitry, a graphics processor, a central processing unit, or combinations thereof. In a score-based gating approach, the gating computation may comprise calculating a linear score from the optical vector and pre-computed weights. The pre-computed weights may be derived from paternal allele information, one or more predictor models, utility preferences, accessibility parameters, or combinations thereof. In some cases, the score provides a rapid approximation that enriches for sperm expected to perform well under a later, more detailed probabilistic inference and ranking stage. The real-time processing hardware may compare the calculated score to a threshold to determine whether a sperm satisfies a selection rule. In a pattern-matching gating approach, the gating computation may comprise comparing the optical vector to one or more stored target patterns. The one or more stored target patterns may correspond to desired haplotype fragments, desirable predicted genetic states, or other target classes. In some cases, the one or more stored target patterns may be maintained in a lookup table or in a hash structure accessible to the real-time processing hardware. In some cases, a sperm may be accepted when the processed optical vector matches one of the stored target patterns or otherwise satisfies a classification rule.

The method 700 may further include a step 706 of combining the gating computation and the optical vector to produce a sorter-control output and a sorter trigger. The sorter-control output and the sorter trigger may generate control signals to actuate a sorting mechanism, such as valve actuation or droplet charging, based on whether the gating computation satisfies a threshold or classification criterion. In some cases, the sorter-control output and the sorter trigger may be generated within less than 100 microseconds. In some cases, the real-time processing hardware may generate the gating computation within less than 100 microseconds, thereby remaining compatible with high-speed sorting. The output of the real-time computation may be a binary accept/reject decision, a ranked class assignment, a score, or a control signal for actuation of a sorting mechanism. The method 700 may support different sorting modes. In some cases, a sorter may operate in a single-pattern mode retaining only sperm meeting a narrow target pattern. In some cases, a sorter may operate in a score-and-store mode collecting sperm exceeding a threshold, such as the top 1% to 10% of interrogated sperm. In the single-pattern mode, sperm matching one of the stored target patterns may be collected. In the score-and-store mode, sperm having scores above a threshold may be collected for later probabilistic ranking. The sorting mode may be selected based on clinical objectives, donor characteristics, panel design, or workflow requirements.

The method 700 may optionally include a step 708 of receiving a calibration input and/or quality control input to refine the gating computation. Calibration and quality-control operations performed before, during, or after a sorting run. In some embodiments, channel-specific error behavior is estimated and supplied to a probabilistic reconstruction model. In some embodiments, locus-specific accessibility or amplification-success behavior is estimated and likewise supplied to a reconstruction model. An emission model may incorporate channel-specific error parameters together with accessibility parameters so that downstream inference and ranking are robust to assay noise and locus-specific measurability rather than assuming error-free optical measurements. In some embodiments, quality metrics are stored together with the indexed sperm record and may be used in later ranking, retrieval, reporting, or audit functions. Thus, the quality-control framework may span the full pipeline, including optical acquisition, real-time gating, indexed storage, post-sort inference, and downstream reproductive use.

The calibration input may comprise updated thresholds, channel-error parameters, or model parameters based on control data or observed population statistics. In some cases, one or more gating thresholds, channel-error parameters, accessibility parameters, amplification-success parameters, or related model parameters may be adjusted in real time or near real time based on updated estimates derived from control data or observed population statistics. Such adaptive updating reduces drift during a run and improves consistency between real-time sorting decisions and post-sort probabilistic inference. The calibration input may allow the sorter to adapt to observed signal quality or channel behavior during a run. In some embodiments, the secondary aliquot 124 of the live sperm sample 120 may be used to generate control data in a pre-run phase, for use as calibration input during step 708. During a sorting run, the control data and observed run data may be used to perform accessibility estimation, channel-error estimation, threshold adjustment, and model updating. These updates may be provided to gating hardware 140 and/or a posterior inference engine 170. In a post-run phase, QC outputs may be used to generate low-confidence flags and/or an audit or report record.

The quality-control input may be derived from the posterior probability distribution itself. By way of example, the probabilistic model may output one or more per-chromosome crossover count estimates, crossover-location summaries, posterior-confidence measures, or related metrics. In some embodiments, such outputs are used to flag low-confidence sperm, low-confidence runs, or assay conditions inconsistent with expected meiotic structure.

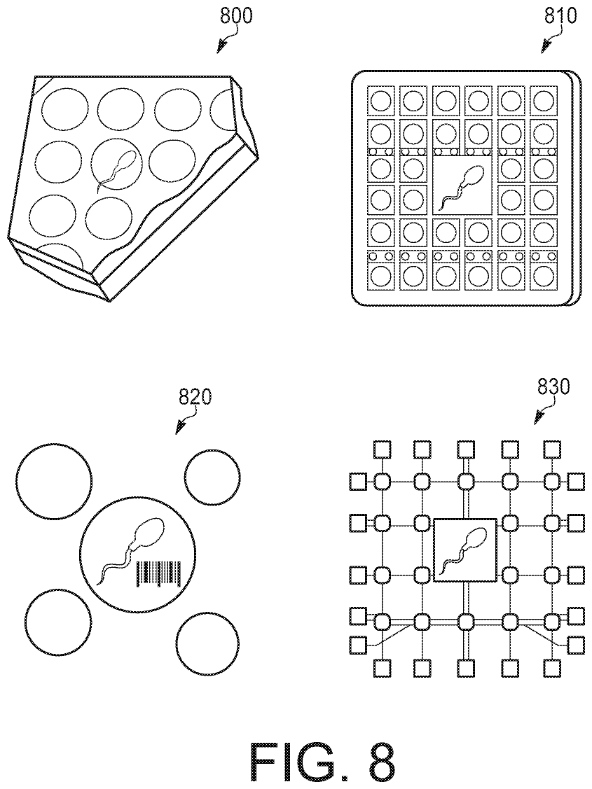

Returning to FIG. 2, the step 210 of method 200 may include a step of collecting the sorted labeled sperm in an indexed collection based on a sorting decision, such as the sorting decision produced according to method 700. As described above with reference to FIG. 1, the indexed collection module 150 may be configured to receive labeled sperm 126 at an indexed collection location based on a sorting decision. The indexed collection location may preserve an association between a physical sperm and corresponding measurement data, including the optical vector 132 and subsequent analysis results. FIG. 8 illustrates exemplary indexed collection formats for indexed collection module 150, including a micro-well plate 800, a nano-well array 810, a droplet or barcoded droplet format 830, or a dielectrophoretic array 840.

Micro-well plate 800 may comprise standard micro-well plates in 96-, 384-, or 1536-format. The micro-well plate 800 may be configured for index sorting, in which individual sperm are directed to specific wells based on sorting decisions. The micro-well plate 800 may have a capacity on the order of 10² to 10³ sperm. In some cases, the micro-well plate 800 may be selected for workflows in which a moderate number of sperm are to be collected and individually tracked. Each well of the micro-well plate 800 may be associated with a unique address or identifier that links the physical sperm to a corresponding digital record including the optical vector and downstream analysis outputs.

Nano-well array 810 may comprise high-density nano-well chips. The nano-well array 810 may have a capacity of about 10⁴ to about 10⁵ sperm. In some cases, the nano-well array 810 may be selected for workflows requiring higher throughput collection while maintaining individual addressability of sorted sperm. The nano-well array 810 may comprise a dense grid of smaller wells, with each well configured to receive and retain an individual sperm. Each nano-well position may be associated with a coordinate or identifier that preserves the link between the physical sperm and corresponding measurement data.

Droplet or barcoded droplet format 820 may comprise droplets configured to encapsulate individual sperm. The droplet or barcoded droplet format 820 may have a capacity of about 10⁵ sperm. In some cases, the droplet or barcoded droplet format 820 may include barcodes associated with individual droplets to preserve the association between each encapsulated sperm and corresponding digital information. The barcodes may be optical barcodes, molecular barcodes, or other identifiers that enable retrieval of measurement data associated with a given sperm. In some cases, the droplet or barcoded droplet format 820 may be selected for high-throughput workflows in which large numbers of sperm are to be collected and tracked.

Dielectrophoretic array 830 may comprise a grid structure with interconnected nodes configured to retain individual sperm using dielectrophoretic forces. The dielectrophoretic array 830 may have a capacity of about 10³ to about 10⁴ sperm. In some cases, the dielectrophoretic array 830 may be selected for workflows in which active manipulation or repositioning of sorted sperm is desired. Each position within the dielectrophoretic array 830 may be associated with an address or coordinate that links the physical sperm to corresponding measurement data.

The indexed collection formats described above may be selected based on desired throughput, retrieval workflow, viability constraints, or clinic-specific handling preferences. In some cases, a run may include multiple collection bins or indexed destinations associated with different thresholds, target classes, or intended downstream uses. The indexed collection formats or surrounding environment may be selected to preserve viability of sorted sperm. In some cases, collection may occur in an appropriate medium, such as human tubal fluid under oil or another viability-preserving medium. In some cases, sperm motility may remain at least about 60% after about 30 minutes in nano-wells at about 37 degrees C. The viability threshold may be selected to ensure that sorted sperm remain suitable for downstream assisted reproductive procedures. In some cases, one or more sorted sperm may be retrieved from indexed collection locations for intracytoplasmic sperm injection, cryopreservation, or further testing. Retrieval may be performed using standard micropipetting, automated micropipetting, or comparable single-cell recovery methods. The retrieval method may be selected based on the indexed collection format, the number of sperm to be retrieved, and the intended downstream use. In some cases, automated micropipetting may be used to retrieve sperm from high-density formats such as the nano-well array 810. In some cases, standard micropipetting may be used to retrieve sperm from the micro-well plate 800 or other formats compatible with manual handling.

Returning to FIG. 2, the method 200 may further include a step 212 of determining a posterior probability distribution for the labeled sperm based on the optical vector and one or more prior inputs. In some cases, determining the posterior probability distribution may comprise using a Hidden Markov Model or an equivalent probabilistic graphical model. The one or more prior inputs may comprise a channel-specific error parameter, an accessibility parameter, paternal haplotype information, or a combination thereof. In some cases, the one or more prior inputs may further comprise the male recombination map. The Hidden Markov Model or equivalent probabilistic graphical model may incorporate the optical vector, the paternal haplotype information, the male recombination map, and the channel-specific error parameters to generate the posterior probability distribution over paternal haplotypes. Step 212 may be performed by posterior haplotype inference engine 170, described above with reference to FIG. 1.

In some embodiments, the posterior haplotype inference engine 170 may be configured to use a Hidden Markov Model or an equivalent probabilistic graphical model. The one or more prior inputs may further comprise channel-specific error parameters and accessibility parameters, as described above. In some cases, the posterior probability distribution may be computed using forward-backward inference or an equivalent algorithm. The output of the forward-backward inference may include posterior probabilities over haplotype states at loci across the genome, one or more inferred crossover locations or crossover-count estimates, and uncertainty measures that may be forwarded to downstream allele-dosage prediction module and genome-wide prediction module.

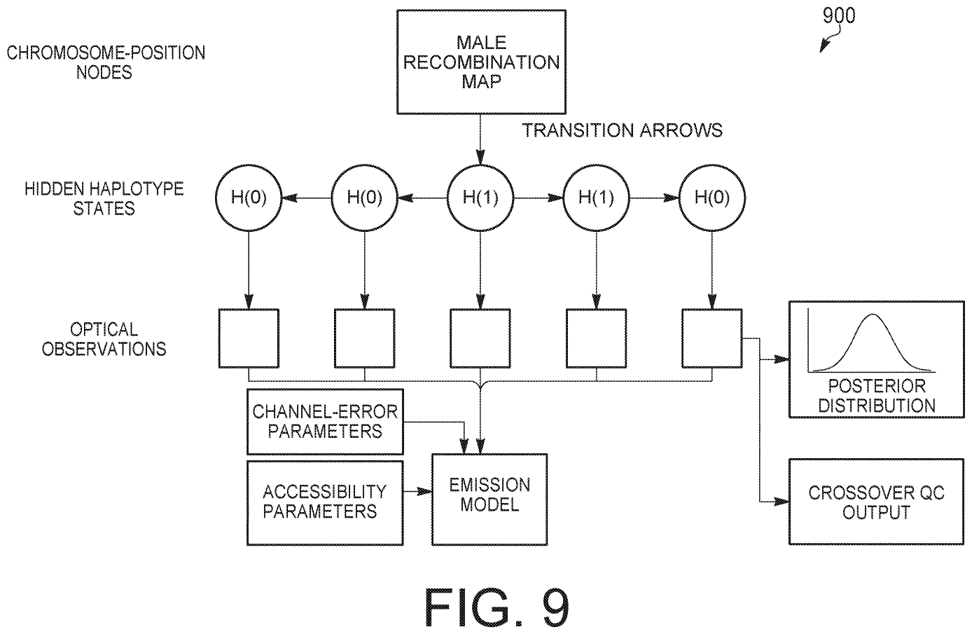

In some embodiments, the posterior haplotype inference engine 170 may utilize a probabilistic reconstruction framework, such as the exemplary probabilistic framework 900 illustrated in FIG. 9. The probabilistic reconstruction framework 900 may be used to reconstruct paternal haplotype origin probabilistically from sparse optical observations and to enable genome-wide inference beyond directly interrogated marker loci. The probabilistic reconstruction framework 900 may include chromosome-position nodes arranged in a sequence. Each chromosome-position node may be associated with hidden haplotype states. The hidden haplotype states may represent paternal haplotype origin along a chromosome. In some cases, the hidden haplotype states may be represented as H(0) and H(1), indicating inheritance from a first paternal haplotype or a second paternal haplotype, respectively. In some cases, the probabilistic reconstruction framework 900 may be evaluated separately by chromosome, by chromosome arm, or across a larger genomic graph structure, provided that the probabilistic reconstruction framework 900 preserves the ability to reason about crossover structure and uncertainty. Transition arrows may connect adjacent hidden haplotype states. The transition arrows may represent the probability of switching between haplotype states based on recombination events (e.g., 30-40 crossover events per male meiosis). Transition behavior in the probabilistic reconstruction framework 900 may be governed by a male recombination map. The probability of switching haplotype origin between adjacent informative positions may be parameterized as a function of recombination mass or genetic distance separating those positions. In some cases, the transition behavior may make posterior inference sensitive to realistic meiotic structure rather than treating.

Optical observations may be associated with respective chromosome-position nodes. The optical observations may feed into an emission model. The emission model may incorporate channel-specific error parameters and accessibility parameters to model the probability of observing a particular optical signal given an underlying allele or haplotype state. In some cases, the channel-specific error parameters may be estimated at about 1% to about 2% mis-call rate per channel. The channel-specific error parameters may account for miscall rates associated with fluorescence detection channels. The accessibility parameters may account for locus-specific measurability in sperm chromatin. In some cases, the emission model may further incorporate amplification-success parameters. The channel-specific error parameters may be estimated from control data, updated from observed run statistics, or carried forward from prior calibration runs. In this manner, the probabilistic reconstruction framework 900 may account explicitly for assay noise and locus-specific measurability rather than treating measured optical states as error-free genotype calls. The probabilistic reconstruction framework 900 may generate a crossover QC output. The crossover QC output may provide per-chromosome crossover count estimates or crossover-location summaries for quality control purposes. In some cases, the crossover QC output may be used to flag low-confidence sperm or assay conditions inconsistent with expected meiotic structure. In some cases, quality metrics derived from the crossover QC output may be stored together with an indexed sperm record and may be used in later ranking, retrieval, reporting, or audit functions.

Techniques for probabilistic inference over inheritance vectors, including Forward-Backward algorithms, Baum-Welch algorithms for estimating phasing error rates, Viterbi algorithms for determining most likely inheritance vectors, and techniques for correcting phasing errors in estimated parental haplotypes, are described in U.S. Patent Application Publication No. 2025/0034645, the entire contents of which are incorporated herein by reference. Methods for probabilistic genomic prediction of sperm genetic content using recombination models and selection criteria for sperm in assisted reproduction, including polygenic scores, chromosomal aneuploidy assessment, and monogenic disorder screening, are described in U.S. Patent Application Publication No. 2025/0034655, the entire contents of which are incorporated herein by reference.

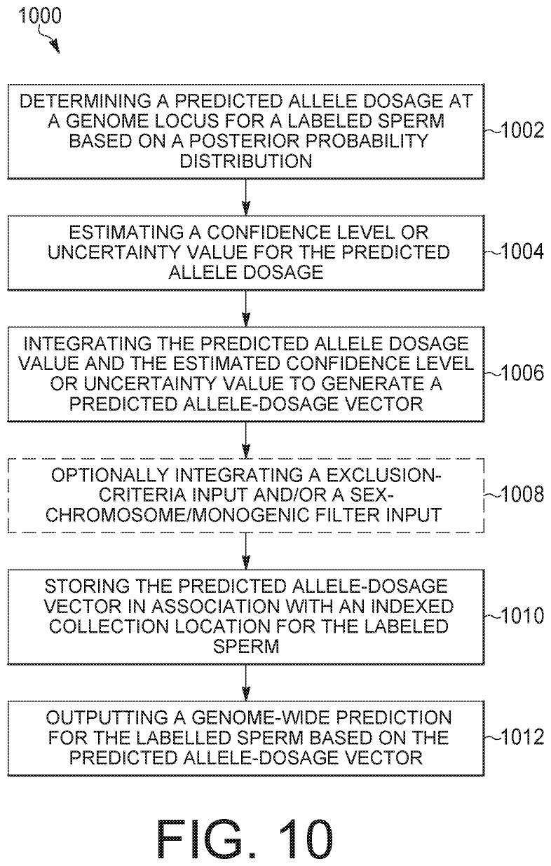

Returning to FIG. 2, the method 200 may further include a step 214 of determining a genome-wide statistical trait prediction based on the posterior probability distribution. The genome-wide statistical trait prediction may include predictions for cardio-metabolic traits, renal and respiratory disease traits, neuropsychiatric and neurodegenerative traits, inflammatory and immunologic traits, cancer-related traits, musculoskeletal and endocrine traits, ocular traits, anthropometric traits, cognitive and behavioral traits, or cardiorespiratory-fitness traits, or other quantitative biomarkers. For example, the genome-wide statistical trait prediction may include a polygenic score, a disease-risk estimate, a liability estimate, a breeding-value estimate, or a combination thereof. Step 214 may be performed by genome-wide prediction engine 180, described above with respect to FIG. 1. Step 214 may further include the steps of method 1000, illustrated by FIG. 10, for determining a genome-wide prediction for a labeled sperm 1000.

As shown in FIG. 10, method 1000 may include the step 1002 of determining a predicted allele dosage at a genome locus for a labeled sperm based on a posterior probability distribution 1002. In some embodiments, the predicted allele dosage may be calculated genome-wide from the inferred paternal haplotype structure rather than being limited to the directly interrogated marker loci. In this manner, sparse optical observations at a selected donor-specific marker panel may be converted into broader genomic predictions for an individual sperm. In some embodiments, the predicted allele dosage may be provided as an input to one or more statistical trait predictor models. Such predictors may include polygenic scores, disease-risk estimates, liability estimates, quantitative trait estimates, breeding-value estimates, or other genome-informed predictors. In some embodiments, a separate predictor is computed for each of a plurality of traits or outcomes of interest. In some embodiments, the same predicted allele dosage may be used to evaluate multiple predictors in parallel for a given sperm.

The method 1000 may further include a step 1004 of estimating a confidence level or uncertainty value for the predicted allele dosage. Confidence intervals or other posterior-derived uncertainty estimates (i.e., uncertainty values) may be obtained from the posterior haplotype distribution, including by sampling-based procedures or other propagation of uncertainty from the reconstruction model into the downstream predictor calculation. In some embodiments, these uncertainty measures are retained with the predictor outputs and are later used in quality control, ranking, or decision making.

The method 1000 may further include a step 10006 of integrating the predicted allele dosage value and the estimated confidence level or uncertainty value to generate a predicted allele-dosage vector. The predicted allele-dosage vector may represent a compact digital representation of the genetic assessment of an individual sorted sperm. In some cases, the same predicted allele-dosage vector may be used to evaluate multiple predictors in parallel for a given sperm.

The method 1000 may optionally include a step 1008 of integrating an exclusion-criteria input and/or a sex-chromosome/monogenic filter input. In some embodiments indexed sperm are ranked according to a utility function. The utility function may be based on one or more predicted traits, one or more disease-risk estimates, one or more uncertainty measures, or combinations thereof. In some embodiments, the utility function corresponds to a clinical utility function used to prioritize sperm expected to provide a preferred downstream outcome in assisted reproduction. In some embodiments, the utility function further includes one or more exclusionary criteria. By way of example, sperm may be deprioritized or excluded if predicted to carry a specified monogenic risk allele, a specified chromosome state, a specified sex-chromosome state, or another undesired genetic feature. In some embodiments, ranking is therefore not limited to maximizing a single statistical trait predictor, but instead reflects a composite decision framework combining predicted benefit, uncertainty, and exclusion rules. In some embodiments, the output of the ranking stage comprises a single selected sperm, a highest-ranked subset of sperm, a thresholded subset, or a ranked list of candidate sperm for downstream use. By way of example, the highest-ranked sperm or a top-k subset may be selected for intracytoplasmic sperm injection, retained for cryopreservation, or stored in indexed locations for later single-cell retrieval. In some embodiments, a ranked set of sperm is retained so that final clinical selection may consider logistical, embryologic, or later-stage testing constraints in addition to the utility score itself. The exclusion-criteria input may be used to exclude sperm predicted to carry a specified monogenic risk allele. In some cases, sperm may be deprioritized or excluded if predicted to carry a specified monogenic risk allele, a specified chromosome state, or another undesired genetic feature. A sex-chromosome filter input may be used to filter sperm based on a sex-chromosome state.

The method 1000 may further include a step 1010 of storing the predicted allele-dosage vector in association with an indexed collection location for the labeled sperm. The stored record may include the predicted allele-dosage vector, the optical vector, the posterior distribution, and any quality-control outputs. In some cases, the method 1000 may further comprise determining a per-chromosome crossover count estimate for quality control. The per-chromosome crossover count estimate may be stored together with the indexed sperm record and may be used in later ranking, retrieval, reporting, or audit functions.

The method 1000 may further include a step 1012 of outputting a genome-wide prediction for the labeled sperm based on the predicted allele-dosage vector. The genome-wide prediction may comprise a polygenic score, a disease-risk estimate, a liability estimate, or a breeding-value estimate. In some cases, a separate predictor may be computed for each of a plurality of traits or outcomes of interest.

### Downstream Applications and Uses

The genome-wide trait predictions and indexed sperm collection produced by the methods and systems described herein may be applied to various downstream application and uses, including utility-based ranking for assisted reproduction, embryo-stage testing, and other uses.

In some embodiments, the methods and systems described herein may be used in a method for ranking a plurality of labeled sperm according to a utility function based on respective genome-wide predictions. The utility function may consider one or more predicted traits, disease-risk estimates, confidence measures, or crossover-related quality-control information. In some cases, the utility function may be scalar, vector-valued, threshold-based, rule-based, or lexicographic in structure. A scalar utility function may produce a single numerical score for each sperm. A vector-valued utility function may produce multiple scores corresponding to different traits or objectives. A threshold-based utility function may apply one or more threshold criteria to determine acceptance or rejection. A rule-based utility function may apply a set of decision rules to determine ranking or selection. A lexicographic utility function may rank sperm according to a prioritized sequence of criteria, where a second criterion is considered only when sperm are tied or equivalent under a first criterion. In some cases, the utility function may further include one or more exclusionary criteria. Sperm may be deprioritized or excluded if predicted to carry a specified monogenic risk allele or a specified sex-chromosome state. In this manner, ranking may reflect a composite decision framework combining predicted benefit, uncertainty, and exclusion rules. In some cases, the output of the ranking stage may comprise a single selected sperm, a highest-ranked subset of sperm, a thresholded subset, or a ranked list of candidate sperm for downstream use. The highest-ranked sperm or a top-k subset may be selected for intracytoplasmic sperm injection, retained for cryopreservation, or stored in indexed locations for later single-cell retrieval.

Sperm cells may be selected for various application based on the utility ranking. In some embodiments, the selection objective is framed as reducing predicted liability to, or improving predicted outcome or value for, one or more complex diseases, disorders, or continuous traits. Illustrative categories include cardio-metabolic traits, renal and respiratory disease traits, neuropsychiatric and neurodegenerative traits, inflammatory and immunologic traits, cancer-related traits, musculoskeletal and endocrine traits, ocular traits, anthropometric traits, cognitive and behavioral traits, cardiorespiratory-fitness traits, and other quantitative biomarkers. In some embodiments, the selection objective instead or additionally includes exclusion of specified monogenic alleles, selection with respect to sex-chromosome state, retention of a ranked candidate set rather than a single selected sperm, or use of the disclosed framework primarily for triage and enrichment before downstream embryo-stage testing. In some embodiments, the signal-generation architecture is direct, indirect, amplified, or hybrid. Thus, the same architecture may be configured for different reproductive, clinical, or breeding objectives without departing from the scope of the present disclosure.

In some embodiments, the methods and systems described herein may be used in a method for assisted reproduction. The method may include selecting a labeled sperm according to a utility-based ranking as described above, and retaining the selected sperm in an indexed collection location suitable for single-cell retrieval, such as nano-wells or barcoded droplets, and are subsequently recovered for use in intracytoplasmic sperm injection, cryopreservation, or related workflows. In some embodiments, retention in indexed locations preserves the link between the physical sperm and its computational ranking so that embryology personnel may retrieve sperm according to the ranking results. In some embodiments, a ranked subset of sperm is retained rather than only a single sperm. For example, the highest-ranked sperm or top-k sperm may be used to fertilize one or more oocytes, thereby preserving flexibility for downstream embryo development, embryo testing, or clinic-specific decision making. In some embodiments, the sperm-stage ranking acts as an earlier selection layer within a broader ART workflow, reducing the number of candidate sperm advanced to downstream procedures while preserving the option of later embryo-stage refinement.

In some embodiments, the methods and systems described herein may be used in a method for embryo-stage testing. The method may include fertilizing one or more oocytes with selected sperm, culturing embryos, genotyping each embryo, and selecting an embryo for transfer based on a second-stage statistical trait predictor that merges paternal posterior information with embryo genotype data. In some cases, embryos derived from selected sperm may undergo one or more forms of preimplantation genetic testing, including aneuploidy testing, polygenic prediction, monogenic testing, or combinations thereof. The method may further include combining sperm-stage paternal posterior information with embryo genotype data and optionally maternal data to generate a refined embryo ranking. The refined embryo ranking may be based on a second-stage statistical trait predictor that merges embryo genotype information with the paternal posterior information generated from the selected sperm. In this two-stage workflow, the sperm-stage process may function as a first selection layer that narrows the fertilization set before embryo-stage testing is performed. The embryo-stage analysis may then provide a later refinement layer that accounts for embryo genotype information, embryo-stage quality metrics, or both, thereby supporting selection of an embryo for transfer based on merged gamete-stage and embryo-stage information.

The present disclosure is not limited to any particular species, clinical setting, or use objective. Accordingly, in some embodiments the disclosed methods are used in human assisted reproduction, and in some embodiments they are used in veterinary breeding or other non-human reproductive settings.

### Computer Implementations

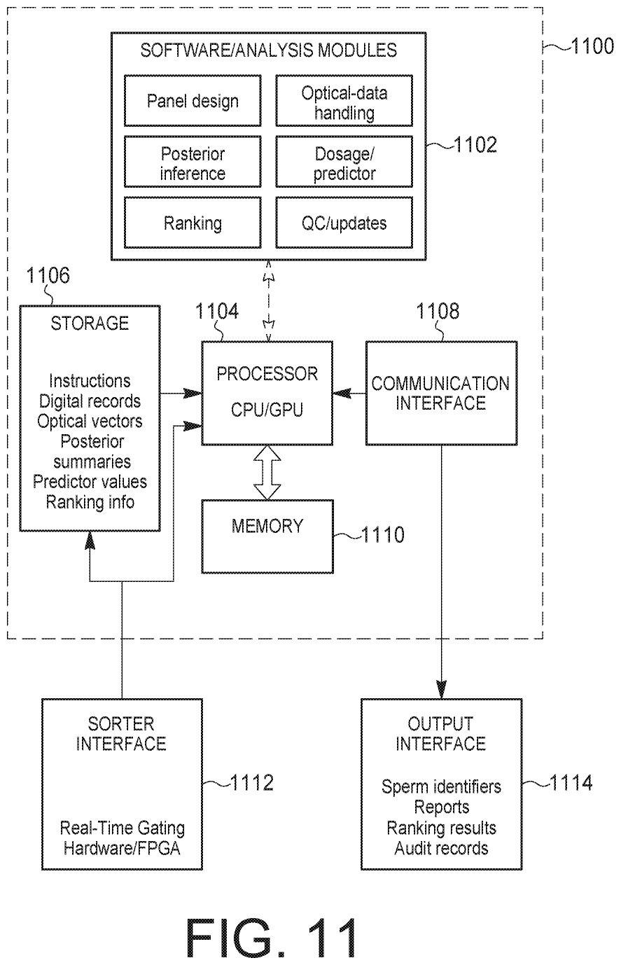

FIG. 11 illustrates a computing environment 1100 configured to perform one or more operations associated with probabilistic single-sperm genome reconstruction and selection. The computing environment 1100 may be used to execute computational operations for marker selection, optical-data management, posterior inference, genome-wide predictor calculation, ranking, control-signal generation, calibration, or data storage.

The computing environment 1100 may include software/analysis modules 1102, a processor 1104, storage 1106, a communication interface 1108, memory 1110, a sorter actuator interface 1112, and an output interface 1114. The processor 1104 may comprise a central processing unit (CPU), a graphics processing unit (GPU), or a combination thereof for executing computational operations. In some cases, post-sort analysis may be performed on a host CPU, GPU, or comparable compute platform using stored optical vectors for indexed sperm. In some cases, real-time gating may be implemented on dedicated hardware associated with a sorter, while posterior inference, genome-wide trait prediction, and ranking may be executed on one or more separate processors.

The software/analysis modules 1102 may include functional blocks for panel design, optical-data handling, posterior inference, dosage or predictor calculations, ranking, and QC or update operations. The software/analysis modules 1102 may be executed by the processor 1104 to perform one or more of the computational operations described herein. In some cases, the software/analysis modules 1102 may be implemented in software, firmware, hardware, or a combination thereof. The partitioning of functions among processors, dedicated circuitry, and storage systems may vary, provided that the computing environment 1100 remains configured to carry out marker-selection, optical-measurement, probabilistic-reconstruction, trait-prediction, and ranking operations.

The processor 1104 may be coupled to the storage 1106. The storage 1106 may store instructions, digital records, optical vectors, posterior summaries, predictor values, and ranking information. The processor 1104 may also be coupled to the memory 1110. The memory 1110 may provide working memory for executing the software/analysis modules 1102.

The communication interface 1108 may be connected to the processor 1104 and may facilitate data exchange with external systems or networks. The communication interface 1108 may enable transmission of data to or from remote computing resources, laboratory information systems, or other external platforms.

The sorter actuator interface 1112 may be coupled to the processor 1104. The sorter actuator interface 1112 may include real-time gating hardware or circuitry configured to generate sorting decisions compatible with high-throughput sperm sorting operations. In some cases, the sorter actuator interface 1112 may comprise a field-programmable gate array (FPGA). The sorter actuator interface 1112 may receive optical vector data and may generate sorting control signals based on gating computations.

The output interface 1114 may be connected to the processor 1104. The output interface 1114 may provide outputs including sperm identifiers, reports, ranking results, and audit records. The output interface 1114 may enable communication of selected sperm identifiers and related information for downstream assisted reproductive technology workflows.

The computing environment 1100 may operate such that the processor 1104 executes the software/analysis modules 1102 using data retrieved from the storage 1106 and the memory 1110. The sorter actuator interface 1112 may receive optical vector data and may generate sorting control signals, while the communication interface 1108 and the output interface 1114 may facilitate data input and output operations for the computing environment 1100.

Digital records generated by the computing environment 1100 may include optical vectors, gating outputs, indexed collection identifiers, posterior summaries, expected allele dosages, predictor values, confidence measures, crossover-related quality-control outputs, and final ranking information. In some cases, the digital records may be stored in the memory 1110, in the storage 1106, or in other computer-accessible data stores. The digital records may be retrieved to support embryology workflows, audit functions, reporting, later model execution, or embryo-stage integration.

In some cases, a non-transitory computer-readable medium may store instructions that, when executed by one or more processors, cause the one or more processors to perform operations comprising one or more of the methods described herein. The instructions may cause the one or more processors to determine a posterior probability distribution over paternal haplotypes for each sorted sperm, derive expected allele dosage at a plurality of loci, calculate at least one genome-wide statistical trait predictor with a confidence interval, rank sperm according to a utility function, and output one or more identifiers corresponding to selected sperm. In some cases, the instructions may further support calibration, quality control, threshold updating, accessibility updating, and data management for indexed sperm records.

### EXAMPLES

The following examples are illustrative and non-limiting. Unless expressly stated otherwise, the examples may be prophetic and are provided to illustrate representative implementations of the disclosed systems and methods.

### Example 1: End-to-End Pre-Fertilization Ranking and Embryo Refinement

In a prophetic example, phased whole-genome sequence data for a sperm donor are processed to identify paternal heterozygous loci, and a donor-specific 40-marker panel is selected to maximize haplotype information content across chromosomes under a male recombination map. Forty allele-discriminating reagents corresponding to the selected loci are prepared, each reagent being associated with a fluorophore resolvable by a multi-color flow cytometer.

A sperm population comprising approximately 10 million sperm is contacted with the 40-plex panel under live-cell conditions and processed through a multi-color sorter configured to generate a fluorescence vector for each sperm. Real-time gating hardware identifies sperm exceeding a threshold score, and the top 0.01% of the interrogated sperm, corresponding to approximately 1,000 sperm, are collected into indexed nano-wells.

For each collected sperm, a probabilistic reconstruction module determines a posterior probability distribution over paternal haplotypes using a Hidden Markov Model that incorporates the fluorescence vector, paternal phased haplotypes, a male recombination map, and channel-specific error parameters. Expected allele dosage is then derived genome-wide, and one or more genome-wide statistical trait predictors are calculated for each collected sperm. The collected sperm are ranked according to a utility function, and the top 50 sperm are selected for ICSI in a modeled workflow.

Embryos resulting from fertilization with the selected sperm are cultured to the blastocyst stage. In one modeled implementation, 12 blastocysts are obtained. Embryo-stage testing, including PGT, is then used to confirm euploidy and refine the ranking. A blastocyst with a favorable predicted outcome is selected for transfer.

### Example 2: Exclusion of a Specified Monogenic Risk Allele

In a prophetic example, a donor-specific marker panel is designed to maximize haplotype informativeness while also ensuring that a paternal genomic region associated with a specified monogenic risk allele is inferable with high confidence from the selected marker set. A live sperm sample is incubated with allele-discriminating reagents corresponding to the selected heterozygous loci and is then processed through a multi-color sorter to generate fluorescence vectors for individual sperm. Real-time gating enriches for sperm predicted to have favorable genome-wide profiles, and accepted sperm are collected into indexed collection locations. For each collected sperm, posterior haplotype inference is performed using a probabilistic graphical model incorporating a sex-specific recombination map and channel-specific error parameters. The posterior distribution is used both to calculate one or more genome-wide statistical trait predictors and to estimate the probability that the sperm carries the specified monogenic risk allele. A utility function is then applied in which sperm having posterior probabilities above a selected threshold for carrying the specified risk allele are excluded from further consideration. The remaining sperm are ranked based on one or more desired statistical trait predictors and associated uncertainty measures. One or more highest-ranked sperm that satisfy the monogenic exclusion rule are retained for fertilization or cryopreservation.

### Example 3: Sex-Chromosome Filtering with Concurrent Autosomal Ranking

In a prophetic example, a donor-specific panel includes a plurality of autosomal haplotype-informative reagents together with at least one reagent directed to a Y-chromosome locus. A live sperm sample is contacted with the panel and processed through a multi-color cytometer to measure fluorescence vectors for individual sperm. Real-time processing hardware classifies sperm according to fluorescence patterns consistent with the presence or absence of a Y-chromosome signal while simultaneously generating a score related to autosomal haplotype informativeness. Sperm satisfying a desired sex-chromosome state are diverted into indexed collection locations, and fluorescence vectors associated with the collected sperm are retained for post-sort inference. For each collected sperm, posterior paternal haplotype reconstruction is carried out using a probabilistic model incorporating the fluorescence observations, paternal phased haplotypes, a male recombination map, and channel-specific error parameters. Expected allele dosages are derived genome-wide, and one or more statistical trait predictors are calculated for sperm within the selected sex-chromosome class. A utility function then ranks sperm within that class, allowing a clinic to select one sperm, a top-k subset, or a retained ranked set for later ICSI or cryopreservation.

### Example 4: Comparison of Score-Based and Pattern-Matching Gating Modes

In a prophetic example, the same donor-specific panel is used in two sorting runs. In a first run, spectrally processed optical vectors are converted to a binarized or ternary representation, and an FPGA computes a linear score using pre-computed weights associated with the measured channels. Sperm having scores above a threshold are collected in a score-and-store mode, for example retaining a top fraction of the sperm population for later probabilistic ranking. In a second run, the same or comparable optical vectors are compared against a set of pre-determined target patterns stored in a lookup structure accessible to the FPGA. Sperm matching one of the target patterns are collected in a single-pattern mode. In both runs, the gating decision is made within a latency budget compatible with high-speed sorting, such as less than 100 microseconds. Post-sort analysis is then performed on collected sperm from both runs using the same probabilistic haplotype-reconstruction pipeline. The resulting posterior distributions, expected allele dosages, statistical trait predictors, uncertainty measures, and crossover-related quality metrics are compared to determine whether score-based enrichment or pattern-based enrichment provides superior downstream ranking performance for the particular donor, panel design, and clinical objective.

### Example 5: Two-Stage Sperm and Embryo Selection Workflow

In a prophetic example, a donor-specific panel is designed from phased paternal genomic data and a male recombination map, and a live sperm sample is interrogated and sorted according to the methods described herein. Accepted sperm are collected into indexed locations, and for each accepted sperm a posterior haplotype distribution is computed and converted into genome-wide expected allele dosages and one or more statistical trait predictors. A ranked subset of the accepted sperm is selected for fertilization of multiple oocytes. Embryos produced from those fertilization events are cultured and genotyped. Embryo genotype data are then combined with the sperm-stage paternal posterior information and, optionally, maternal genomic information to generate a second-stage statistical trait predictor or refined embryo ranking. In this two-stage workflow, the sperm-stage process functions as a first selection layer that narrows the fertilization set before embryo-stage testing is performed. The embryo-stage analysis then provides a later refinement layer that may account for embryo genotype information, embryo-stage quality metrics, or both, thereby supporting selection of an embryo for transfer based on merged gamete-stage and embryo-stage information.

### Example 6: Adaptive Threshold and Error-Model Updating During a Sorting Run

In a prophetic example, a live sperm sample labeled with a donor-specific panel is processed through a multi-color sorter equipped with real-time gating hardware and a host compute system. During the run, control data and observed population statistics are used to estimate channel-specific error behavior, including miscall tendencies associated with particular fluorescence channels. One or more gating thresholds and one or more channel-error parameters used in downstream haplotype reconstruction are updated in real time or near real time based on the revised estimates. Accepted sperm are collected into indexed locations under the updated gating regime, and posterior haplotype inference is performed using the updated error parameters. The resulting sperm rankings are compared with rankings obtained under a fixed-parameter regime to assess robustness, posterior confidence, and consistency with expected crossover structure. This adaptive workflow may improve agreement between real-time sorting decisions and later probabilistic inference, particularly in long runs or in runs exhibiting optical drift, changing signal quality, or donor-specific assay variability.

### Example 7: Chromatin-State-Aware Panel Design with Calibration-Aliquot Refinement

In a prophetic example, phased paternal genomic data are intersected with one or more accessibility priors derived from retained-nucleosome annotations, GC-content profiles, promoter annotations, and other chromatin-state features. Candidate heterozygous loci satisfying recombination-spacing constraints are ranked using a combined informativeness-and-accessibility objective. A preliminary reagent panel is then tested on a sacrificial calibration aliquot from the same donor. Locus-specific signal intensity, detection rate, and background are measured, and empirical accessibility estimates are used to update the panel design. A refined final panel is selected that maximizes haplotype information content while preferentially retaining loci that are empirically measurable in the donor's sperm chromatin.

### Example 8: Indirect Signal Generation by Secondary Reporter or Localized Amplification

In a prophetic example, a first allele-discriminating reagent binds or creates a structural opening at a selected locus in an allele-specific manner but does not itself provide the primary fluorescence signal. A secondary reporter or amplification chemistry is then introduced to generate a localized fluorescent signal at the targeted locus. By way of example, a padlock probe may hybridize or ligate in association with the allele-discriminating event, after which rolling circle amplification, hybridization chain reaction, branched-DNA amplification, or another localized amplification chemistry produces a multi-copy fluorescent signal that is measured by the sorter. In some embodiments, indirect signal generation allows loci that would otherwise produce insufficient direct fluorescence to remain usable within the panel.

A number of implementations have been described. Nevertheless, it will be understood that various modifications may be made without departing from the spirit and scope of the disclosure. Accordingly, other implementations are within the scope of the following claims.

1. A method for determining a genome-wide statistical trait prediction from a sperm cell, the method comprising:
obtaining a primary aliquot of a live sperm sample;
incubating the primary aliquot with an allele-discriminating reagent panel to produce a labeled sperm, wherein the allele-discriminating reagent panel comprises a plurality of allele-discriminating reagents respectively directed to heterozygous markers selected based on phased paternal genomic information, a male recombination map, and one or more constraints;
detecting an optical signal from the labeled sperm using an optical interrogation instrument;
determining an optical vector based on the optical signal;
sorting the labeled sperm to an indexed collection location, wherein the indexed collection location preserves an association between the labeled sperm and a digital record including the optical vector;
determining a posterior probability distribution for the labeled sperm based on the optical vector and one or more prior inputs; and
determining a genome-wide statistical trait prediction based on the posterior probability distribution.

2. The method of claim 1, wherein the one or more constraints includes a chromatin-accessibility prior, a fluorescence-channel constraint, a recombination-based spacing constraint, or a combination thereof.

3. The method of claim 1, wherein the allele-discriminating reagent panel comprises an allele-discriminating reagent comprising a target-binding region configured to bind to a target locus, and wherein the allele-discriminating reagent is configured to generate an optical signal.

4. The method of claim 1, wherein sorting the labeled sperm comprises determining a gating computation for the labeled sperm, and wherein the gating computation is score-based or pattern-based.

5. The method of claim 1, wherein the indexed collection location comprises a micro-well plate, a nano-well array, a droplet or barcoded droplet format, or a dielectrophoretic array.

6. The method of claim 1, wherein determining the posterior probability distribution comprises using a Hidden Markov Model or an equivalent probabilistic graphical model.

7. The method of claim 6, wherein the one or more prior inputs comprise a channel-specific error parameter, an accessibility parameter, paternal haplotype information, or a combination thereof.

8. The method of claim 1, further comprising predicting an expected allele dosage at a plurality of loci based on the posterior probability distribution.

9. The method of claim 1, further comprising determining a confidence interval or uncertainty estimate for the genome-wide statistical trait prediction.

10. The method of claim 1, further comprising selecting the labeled sperm for fertilization according to a utility function that ranks the genome-wide statistical trait prediction.

11. A system for determining a genome-wide statistical trait prediction from a sperm cell, the system comprising:
an optical interrogation instrument configured to detect an optical signal from a labeled sperm and to generate an optical vector based on the optical signal, wherein the labeled sperm is produced by incubating a sperm cell with an allele-discriminating reagent panel comprising a plurality of allele-discriminating reagents respectively directed to a set of heterozygous markers;
real-time gating hardware configured to receive the optical vector and to generate a sorting decision based on the optical vector;
an indexed collection module configured to receive the labeled sperm at an indexed collection location based on the sorting decision, wherein the indexed collection location preserves an association between the labeled sperm and a digital record including the optical vector; and
a processor configured to perform operations comprising:
obtaining a primary aliquot of a live sperm sample;
incubating the primary aliquot with an allele-discriminating reagent panel to produce a labeled sperm, wherein the allele-discriminating reagent panel comprises a plurality of allele-discriminating reagents respectively directed to heterozygous markers selected based on phased paternal genomic information, a male recombination map, and one or more constraints;
detecting an optical signal from the labeled sperm using an optical interrogation instrument;
determining an optical vector based on the optical signal;
sorting the labeled sperm to an indexed collection location, wherein the indexed collection location preserves an association between the labeled sperm and a digital record including the optical vector;
determining a posterior probability distribution for the labeled sperm based on the optical vector and one or more prior inputs; and
determining a genome-wide statistical trait prediction based on the posterior probability distribution.

12. The system of claim 11, wherein the optical interrogation instrument comprises a multi-color flow cytometer or an optical-imaging flow cytometer.

13. The system of claim 11, wherein the real-time gating hardware comprises a field-programmable gate array.

14. The system of claim 11, wherein the real-time gating hardware is configured to generate the sorting decision within less than or equal to 100 microseconds.

15. The system of claim 11, wherein the indexed collection module comprises a micro-well plate, a nano-well array, a droplet format, a barcoded droplet format, or a dielectrophoretic array.

16. The system of claim 11, wherein the posterior probability distribution is configured to use a Hidden Markov Model or an equivalent probabilistic graphical model.

17. The system of claim 11, wherein the one or more prior inputs further comprise channel-specific error parameters and accessibility parameters.

18. The system of claim 11, further comprising a genome-wide prediction engine configured to derive an expected allele dosage at a plurality of loci and at least one genome-wide statistical trait prediction based on the posterior probability distribution.

19. The system of claim 18, further comprising a utility-ranking module configured to rank a plurality of labeled sperm according to a utility function based on respective genome-wide statistical trait predictions.

20. The system of claim 11, wherein the set of heterozygous markers are selected based on the phased paternal genomic information, the male recombination map, and one or more constraints.
